(19) United States
(12) Patent Application Publication
(10) Pub. No.: US 2001/0043949 A1
(43) Pub. Date: Nov. 22, 2001

(54) Microparticulate Composition

(75) Inventor: Araceli Delgado, La Laguna (ES)

Correspondence Address: AKIN, GUMP, STRAUSS, HAUER & FELD, L.L.P., ONE COMMERCE SQUARE, 2005 MARKET STREET, SUITE 2200, PHILADELPHIA, PA, 19103, US

(73) Assignee: West Pharmaceutical Services Drug Delivery & Clinical Research Center, Ltd

(21) Appl. No.: 09/791,184

(22) Filed: Feb. 23, 2001

Related U.S. Application Data
(63) Continuation of application No. PCT/GB99/02775, filed on Aug. 23, 1999.

(30) Foreign Application Priority Data
Aug. 27, 1998 (GB) 9818591.1

(51) Int. Cl. A61K 9/16; A61K 9/50

(52) U.S. Cl. 424/491; 424/494; 424/497; 264/4.1

(57) ABSTRACT

A microparticulate composition comprises a biodegradable synthetic polymer microparticle, a proteinaceous antigen and an enteric polymer, wherein the enteric polymer forms a coating layer on a surface of the microparticle.

### CROSS-REFERENCE TO RELATED APPLICATION

This application is a continuation of International Application No. PCT/GB99/02775, filed Aug. 23, 1999, the disclosure of which is incorporated herein by reference.

### BACKGROUND OF THE INVENTION

The present invention relates to a microparticulate composition and more particularly to a microparticulate drug delivery composition in which the drug is a proteinaceous antigen. The compositions of the invention may exhibit enhanced mucosal delivery.

It is known that advantages can be obtained by delivering therapeutic materials such as drugs, diagnostic agents and antigens to specific sites in the body. Various methods and systems have been proposed (see the review by Pettit and Gombotz in Trends in Biotechnology, page 343 (1998)).

Microparticulate carriers in the form of microspheres and microcapsules can be used for the delivery of therapeutic materials into the blood stream, into body tissues or into the body cavities and lumens of the nose, gastrointestinal tract, vaginal cavity, etc. Such microparticulate systems are familiar to those skilled in the art (see for example the book by Davis et al. Microspheres and Drug Therapy, Elsevier, Holland (1984)).

Microparticles can be produced using a range of biodegradable and biocompatible polymers. These polymers can provide particles with different physicochemical characteristics, e.g., size and different degradation rates, as well as different levels of loading of the therapeutic agent. In the field of antigen and drug delivery, polylactide polymers and polylactide-co-glycolide polymers have been popular as materials from which microparticles can be prepared.

The delivery of microparticles containing therapeutic agents to the gastrointestinal tract of vertebrates such as fish and mammals can be advantageous. It has been shown that such particles can be taken up by certain cells that line the gastrointestinal tract, such as the epithelial cells (enterocytes) and specialized cells called M-cells (microfold cells) located in Peyer's patches. The cells of the colon wall, such as colonocytes and lymphoid cells, also represent suitable targets. Similar types of specialized cells are present in the nasal cavity.

The encapsulation of antigens in microparticles for use as oral vaccines has been described in the prior art. A significant proportion of the antigen may be entrapped inside the particle and therefore is not exposed to the external environment in the gastrointestinal tract. However, a further significant proportion, e.g., greater than 60%, of the antigen may be attached to the surface of the particle. Some of the surface-adsorbed material may be released quickly after administration (the so-called burst effect), but a proportion of the surface material can be tightly bound to the particle and is believed to be a critical determinant in the resultant immune response.

When a microparticle carrying an antigenic material is administered to the gastrointestinal tract of a vertebrate, the material incorporated inside the polymer matrix should be protected satisfactorily by that matrix. In contrast, the surface exposed antigenic material can be degraded or modified unfavorably by the effect of endogenous pH and enzymes. Consequently, the vaccine system will be less efficacious.

The oral administration of an active agent to the lymphoid tissue of the small intestine (Peyer's patch) using microcapsules formed from a biodegradable and biocompatible synthetic polymer, such as polylactide-co-glycolide, is described in European published patent application EP-A-0 266 119. The use of an enteric polymer to coat such particles is not described.

The enteric coating of formulations containing whole microorganisms has also been described in the prior art. For example, an early description of enteric coated particles in oral vaccine delivery involved the encapsulation of Escherichia coli heat labile enterotoxin in so-called microspheres (3 mm in diameter) prepared from starch and cellulose with hydroxypropyhnethylcellulose phthalate as the enteric coating polymer (Klipstein et al., Infect. Immun., 39:1000 (1983)). Oral administration of this formulation induced serum and intestinal antibody responses comparable to those induced following oral delivery of the antigen alone after a dose of the gastric inhibitor cimetidine. There was no suggestion that microparticles less than 1000 microns, made from synthetic polymers, could be coated with an enteric layer.

Cellulose acetate phthalate has also been used to coat microspheres of 1-3 mm in size containing a virus (Maharaj et al., J. Pharm. Sci., 73:39 (1984)). The same polymer has also been used to produce microspheres with entrapped bacteria (Lin et al., J. Microencaps., 8:317 (1991)). These different formulations were designed to protect the antigen against degradation in gastric fluid and facilitate its subsequent release in the intestine. There was no suggestion that proteinaceous antigens could be entrapped in microparticles less than 1000 μm and the resulting microparticles enterically coated.

An oral vaccine comprising a live recombinant adenovirus in an enteric-coated dosage form is described in British published paten application GB-A-2 166 349. No mention is made of microparticulate polymeric carriers.

Bender et al., J Virol., 70:6418 (1996), has suggested that a replication-deficient, orally administered enteric coated vaccina virus vectored vaccine might safely protect against influenza. Similarly, Bergmann et al., Int. Arch. Allergy Appl. Immunol. 80:107 (1986), administered an enteric coated inactivated influenza vaccine to 5 volunteers via the oral route. Neither of these systems comprised biodegradable microparticles made from synthetic polymers.

U.S. Pat. No. 5,676,950 describes a recombinant vaccine or pox virus for oral administration, where an enteric coating can be used so that the virus is released only when it reaches the small intestine. There is no description of biodegradable synthetic polymeric microparticles.

Particulate carriers having a solid core comprising a polysaccharide and a proteinaceous material and an organometallic polymer bound to the core as a protective coating are described in International application publication WO-95/31187. There is no description of biodegradable synthetic polymeric microparticles.

Oral compositions of sensitive proteinaceous agents, such as an immunological agent or vaccine, have been disclosed in U.S. Pat. No. 5,032,405. This patent discloses a particulate diluent uniformly coated with an alkaline soluble polymeric coat, which will dissolve at a specific pH. The polymer coat comprises at least one partially esterified methacrylic acid. The particulate diluent comprised maltose and optionally a further material, such as an inorganic salt. No mention is made of a proteinaceous antigen adsorbed on the surface of biodegradable synthetic polymeric microparticles such as those formed from polylactide or polylactide-co-glycolide.

Microspheres with a core layer containing an immunogen and an enteric coating, which protects and retains shape at room temperature, have been described in International application publication WO-98/07443. The enteric coating is soluble in the digestive tract and has the property of maintaining sphere structure at room temperature. The microspheres were prepared from gelatin by extruding an immunogen suspension fluid from the central tube and an aqueous solution of the enteric substance from the outer tube of a concentric multi-tube nozzle into a solution to solidify the drops. Microspheres prepared from synthetic biodegradable polymers were not described.

International application publication WO-92/00096 describes an oral vaccine composition that can be formulated as enteric dosage forms in the form of microspheres, biodegradable microcapsules or liposomes. Enteric coatings are not described.

Oral pig vaccines as enteric-coated microparticles having a globular shape and critical maximum diameter are disclosed in German patent publication DE 23 43 570. The particles have a diameter of preferably less than 1.5 mm and are coated with cellulose acetate phthalate. The core is a solid carrier such as barium sulphate. Synthetic polymer carriers are not described.

Gelatin spheres coated with an enteric film for oral administration of immunogen are described in Japanese patent publication JP-5-294845. Polymeric microparticles produced from synthetic polymers were not described.

U.S. Pat. No. 5,591,433 describes the microencapsulation of a protein with an aqueous solution of an enteric polymer. The protein, which can be an immunogen, is not attached to or incorporated in a polymeric microparticle. Indeed, the objective in U.S. Pat. No. 5,591,433 is to allow the release of the protein into solution in the intestine to avoid degradation of the protein in the stomach.

### BRIEF SUMMARY OF THE INVENTION

Microparticulate oral drug delivery compositions comprising biodegradable polymeric microparticles prepared from synthetic polymers, a proteinaceous antigen encapsulated by and surface adsorbed on the microparticles and a protective coating of an enteric polymer over the surface of the microparticles have not been previously described. Furthermore, the preparation of such microparticles by a water-in-oil-in-water double emulsion process in which the enteric polymer is used as the stabilizing agent has not been previously described.

We have now developed a biodegradable microparticulate drug delivery composition, which is adapted for oral administration, in which the microparticles carry a surface layer of an enteric polymer that protects surface-adsorbed antigen from degradation or modification in the gastrointestinal tract and particularly the stomach of an animal. The protective coating of the enteric polymer can lead to an improved immune response when the microparticles are administered orally to an animal.

By “biodegradable” is meant a material that can degrade upon administration to a living organism, such as a mammal or fish. The degradation may be through the non-specific cleavage of chemical bonds, such as hydrolysis of an ester, or through an enzyme-catalyzed process. The degradation results in the synthetic polymer decreasing in molecular weight so that the polymeric microparticle eventually dissolves and is no longer resident in the body as an intact particle.

For the case of biodegradable microparticles in the form of microspheres or microcapsules, these can degrade over a period of days, weeks, or months depending on their chemical composition and molecular weight. Degradation can be via a process of surface or bulk erosion or a combination of these processes.

An enteric (or gastro-resistant) polymer is defined as a material that does not dissolve in the stomach of an animal at acidic pH values, but when the polymer transits to the intestines, where the pH is higher than that of the stomach, the polymer will start to dissolve. The threshold pH for such dissolution to occur will depend on the chemical nature of the polymer. Typically enteric polymers contain weak acid groups that can ionize at pH values above their pKa values and start to dissolve. A review on enteric polymers by Healy can be found in Drug Delivery to the Gastrointestinal Tract, Chapter 7, Hardy, Davis, Wilson (eds), Ellis Horwood, Chichester (1989).

### BRIEF DESCRIPTION OF THE SEVERAL VIEWS OF DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a bar graph of levels of specific IgA anti-OVA antibodies detected at weekly intervals in serum of mice after booster immunization according to Example 6 below; and

FIG. 2 is a bar graph of levels of specific IgA anti-OVA antibodies detected at weekly intervals in saliva of mice after booster immunization according to Example 6 below.

### DETAILED DESCRIPTION OF THE INVENTION

According to a first aspect of the present invention, there is provided a microparticulate composition comprising a biodegradable synthetic polymer, a proteinaceous antigen and an enteric polymer, wherein the enteric polymer forms a coating layer on the surface of the microparticles.

The microparticulate composition of the invention may be used for delivering drugs. The composition comprises polymeric microparticles which are made from a biodegradable synthetic polymer and which are loaded with the proteinaceous antigen. The enteric polymer forms a coating or layer on the surface of the microparticles.

It will be appreciated that the enteric polymer will not necessarily coat the entire outer surface of the microparticles. Typically, from 40 to 100% of the outer surface of the microparticles will be covered by the enteric polymer. Preferably at least 60% of the surface will be covered and most preferably at least 80% of the surface will be covered.

By a “microparticulate composition” is meant a composition which is comprised of microspheres and/or microcapsules. By a “microparticle” is meant a particle that is less than 1000 μm in diameter comprising a matrix of the biodegradable synthetic polymer which carries the proteinaceous antigen. A particle diameter in the range 0.1 to 20 μm is preferred, more preferably in the range 0.5 to 10 μm and most preferably in the range 1.0 to 5.0 μm. The antigen may be dispersed within the microsphere, on the surface of the microsphere or more typically will be divided between these two locations. Such surface adsorbed antigen can be important to the correct presentation of the antigen to the cells of the immune system.

By a “microcapsule” is meant a hollow or voided particle which comprises one or more hollows or voids which are surrounded by a matrix formed from the biodegradable synthetic polymer. The proteinaceous antigen is located in the hollow or void(s) of the capsule and on its surface. In one particular embodiment, the microcapsule comprises a centrally located hollow which contains a proportion of the proteinaceous antigen and a surrounding shell or casing which is formed from the biodegradable synthetic polymer.

Whether the microparticle is a microsphere or a microcapsule, the enteric polymer forms a coating on the outer surface of the particle and protects surface-adsorbed antigen from degradation or modification.

Microparticles for the improved delivery of antigens can be made from synthetic biodegradable polymers using methods known in the art, such as emulsification, phase separation and spray drying (see the article by Kissel et al. in Antigen Delivery Systems, Chapter 10, Gander et al.(eds.) Harwood Academic Publishers, Netherlands (1997)).

In the spray drying process, the material used to form the body of the microparticles is dissolved in a suitable solvent (usually water), and the solution is spray dried by passing it through an atomization nozzle into a heated chamber. The solvent evaporates to leave microparticles.

Preferred emulsification methods are the water-in-oil-in-water and the water-in-oil-in-oil double emulsification methods.

The water-in-oil-in-water double emulsification method involves the preparation of a water-in-oil-in-water emulsion. The antigen is dissolved in water or an aqueous solution containing a buffer and/or other formulation components, such as sugars, cyclodextrins, etc. The aqueous solution of the antigen is then emulsified in an immiscible oil phase, comprising an organic solvent in which the biodegradable synthetic polymer is dissolved, to produce a water-in-oil emulsion (w/o). A stabilizing agent can be used in the preparation of this initial w/o emulsion. The choice of organic solvent will be dictated by the properties of the biodegradable polymer. Suitable solvents include, inter alia, dichloromethane, ethylacetate, ethyl formate and chloroform. The solubility product concept may be used to select an appropriate polymer/solvent combination. The resultant water-in-oil emulsion is then re-emulsified into an aqueous phase to produce a double water-in-oil-in water emulsion (w/o/w). The second (external) aqueous phase contains an agent that will stabilize the double emulsion and the microparticles which are formed, such as polyvinylalcohol (PVA). In a preferred embodiment, the enteric polymer is used as the stabilizing agent in the second aqueous phase (see infra). The organic solvent is then removed by evaporation or extraction resulting in the formation of rigid microparticles where the contents of the internal aqueous phase which include the antigen are entrapped to a lesser or greater extent inside the biodegradable polymer.

The water-in-oil-in-oil method is described in International application PCT/GB95/01426 (Yeh et al.). In this process, an aqueous solution of the material to be encapsulated (e.g., protein) is emulsified with a first organic solvent (e.g., dichloromethane). This water-in-oil emulsion is then mixed with a solution of biodegradable polymer (e.g., poly-L-lactide), dissolved in the same (e.g., dichloromethane) or a second organic solvent. Finally, this mixture is emulsified with a third organic solvent (e.g., methanol), which is miscible with the first and second organic solvents, but is not a solvent for the polymer, to form a water-in-oil-in-oil emulsion. The emulsion is stirred until the dispersed solvent (e.g., dichloromethane) is extracted. The microparticles thus formed are washed several times in water and freeze dried.

Of the above techniques for making microparticles, the water-in-oil-in-oil and especially the water-in-oil-in-water methods are preferred.

The therapeutic antigen is incorporated in or onto the microparticle to a varying degree of efficiency. This can be from less than 0.01% w/w to greater than 40% w/w loading on the total weight of the microparticle depending on the nature of the polymeric material used for the microparticles as well as the properties of the therapeutic antigen and the processing method. The antigen can be loaded onto the microparticles after they have been prepared and isolated providing that this is done before the microparticles are coated with the enteric polymer. Generally, however, the antigen is incorporated during the manufacturing process used to make the microparticles and will tend to collect inside the microparticles as well as being adsorbed on the outer surface of these particles.

The enteric polymer which coats the surface of the microparticles may be applied to already formed microparticles, e.g., prepared as described above, using coating techniques known in the art such as spraying, and dipping.

Thus, in accordance with a second aspect of the present invention, there is provided a process for preparing a microparticulate composition comprising polymeric microparticles formed from a biodegradable synthetic polymer, a proteinaceous antigen carried by the microparticles and a coating of an enteric polymer on the surface of the microparticles, which process comprises forming the polymeric microparticles carrying the antigen and coating the surface of the so formed microparticles with an enteric polymer.

However, it has been discovered that it is possible to produce the microparticles of the present invention by a water-in-oil-in-water emulsion technique in which the enteric polymer is used as a stabilizing agent during the preparation of the microparticles rather than the more usual stabilizing agents such as polyvinyl alcohol. The enteric polymer can preferentially locate at the surface of the microparticles during the manufacturing process in much the same way as conventional stabilizing agents and thereby encourages the formation of discreet, non-aggregated microparticles.

With this technique, microparticles carrying a surface layer of the enteric polymer are prepared in a single step process, so that there is no need to carry out a discrete coating step to apply the enteric layer.

Thus, in accordance with a third aspect of the present invention, there is provided a process for preparing a microparticulate composition comprising polymeric microparticles formed from a biodegradable synthetic polymer, a proteinaceous antigen carried by the microparticles and a coating of an enteric polymer on the surface of the microparticles, which process comprises forming the polymeric microparticles in the presence of the antigen and the enteric polymer. In a preferred embodiment, the process is an emulsification process, particularly a water-in-oil-in-water emulsification process, in which the enteric polymer acts as a stabilizer for the microparticles which are formed in the process.

Suitable biodegradable synthetic polymers for use in the present invention include, but are not limited to, polylactides, polylactide-co-glycolides, polycaprolactones, polyhydroxyalkanoates, polyorthoesters, polyanhydrides, polyphosphazenes, polyalkylcyanoacrylates, polymalic acids, polyacrylamides, polylactide-PEGs, polyethyleneglycol copolymers and polycarbonates. These polymers can be processed to produce rigid microparticles.

Polylactide-co-glycolide is a preferred polymer for the microparticle. The molar ratio of lactide to glycolide can be 10 to 90%. 50:50 and 75:25 mixtures on a molar basis of lactide to glycolide are preferred. The molecular weight of the polylactide-co-glycolide polymer can be 2 kD to 200 kD. A molecular weight of 10 to 50 kD is preferred.

Polylactide is another preferred biodegradable polymer for the microparticles. The molecular weight of this polymer can be 1 kD to 400 kD. A material with a molecular weight in the range 2 to 10 kD is preferred.

Suitable enteric polymers include, inter alia, cellulose acetate trimelletate, hydroxypropylmethylcellulosephthalate, polyvinylactatephthalate, cellulose acetate phthalate, shellac, methacrylic acid copolymers, such as Eudragit L-100-55, which is an anionic copolymer based on methacrylic acid and ethyl acrylate and is described in the United States Pharmacopeia/National Formulary as a methacrylic acid copolymer, type C. Carboxymethylethyl cellulose (CMEC) is a preferred material. Commercially available CMEC has a mean molecular weight of 49 kD. The content of carboxymethyl and ethoxyl groups in the polymer can be in the range of 8.9 to 14.9% and 32 to 43% w/w, respectively.

A proteinaceous antigen is one that is obtained from the surface or core of a virus or is the surface or internal material of a bacterium or parasite. The protein can be a glycoprotein, such as GP120 (known for the HIV virus). Examples of proteinaceous materials include the nuclear proteins of influenza, surface proteins of influenza and pertussis, fimbrial proteins of E. coli toxoid and toxins. The antigen can be prepared from a microorganism or through a process of genetic engineering where a construct (fusion protein) can be grown in a bacterial or mammalian cell, etc. Such constructs can include the antigen together with a material that can improve the performance of the vaccine, such as a cytokine (interleukin) or immunostimmulatory peptide. The proteinaceous antigen can be a component of the diet that may give rise to allergy, such as ovalbumin or proteins from shell fish or peanuts.

By controlling the thickness of the enteric coating it will be possible to deliver the surface attached antigen undamaged to the distal small intestine (ileal region) or to the various parts of the large intestine.

The composition can be delivered bucally, orally, rectally, nasally, conjunctivally, via the genitourinary tract, or via any appropriate method to a mucosal surface of a vertebrate. Oral delivery is preferred.

The microparticles of the present invention will be particularly useful for the oral immunization of animals (for example by addition to the feed) or for fish (administration to aquaculture) and to children who find difficulty in swallowing solid dosage forms, such as tablets and capsules.

The present invention will now be illustrated, but not limited, with reference to the following specific examples.

### EXAMPLE 1

### Preparation of Microspheres with Enteric Polymers and Entrapped Bioactive Agents

Method

An aqueous solution of ovalbumin (OVA) in distilled water (2 ml, 30 mg/ml) was emulsified with 10 ml of a 6% solution of polylactide-co-glycolide (50:50 polylactide:polyglycolide, 34,000 D molecular weight; Boehringer Ingleheim, Ingleheim, Germany) in dichloromethane using a Silverson homogenizer for 2 minutes at high speed (12,000 rpm) to produce a primary water in oil emulsion. This water-in-oil (w/o) emulsion was then emulsified at high speed with a 10% solution of an enteric polymer as stabilizer to produce a water-in-oil-in-water (w/o/w) emulsion. Either carboxymethylethyl cellulose (CMEC, Freund, Japan) or Eudragit L-100-55 (Rohm Pharma, Germany) was used as the enteric polymer, and different concentrations of these polymers were used and were buffered to a final pH of 6. The w/o/w emulsion was stirred for approximately 18 hours at room temperature and pressure to allow solvent evaporation and microsphere formation. The microspheres were isolated by centrifugation, washed and freeze-dried. The microspheres were examined by scanning electron microscopy for surface morphology and size analyzed by laser diffractometry (Malvern-Mastersizer).

Results

The microparticles stabilized using the enteric polymers displayed a spherical shape and smooth surface and were non-porous. The sizes of the microparticles are as shown below as d(50%) μm, d(10%) μm and d(90%) μm which are the sizes obtained by laser diffractometry as percentage undersize.

| | Particle Size | | | |
|---|---|---|---|---|
| | Stabilizer | | | |
| Stabilizer | (%) w/v | d(50%) (μm) | d(10%) (μm) | d(90%) (μm) |
| Eudragit | 2.5 | 1.31 | 0.43 | 4.08 |
| Eudragit | 4 | 0.96 | 0.52 | 2.39 |
| Eudragit | 6 | 0.81 | 0.44 | 4.01 |
| CMEC | 4 | 0.54 | 0.26 | 1.28 |
| CMEC | 6 | 0.56 | 0.26 | 1.07 |
| CMEC | 8 | 0.40 | 0.19 | 1.97 |

### EXAMPLE 2

### Entrapment of Bioactive Materials in Microspheres with Enteric Polymers

Methods

Microspheres stabilized with enteric polymers and containing OVA were prepared as described in Example 1. The OVA was extracted from the microspheres by one of two means: i) microparticles (3-4 mg) were shaken overnight with 1 ml of 0.1 M sodium hydroxide solution; ii) microparticles (10 mg) were suspended in 0.25 ml of 5% aqueous sodium dodecyl sulphate solution and shaken for 1 hour, then 1 ml of 50:50 dichloromethane:acetone was added and the sample stirred overnight to evaporate the organic solvents. These samples were then analyzed for OVA content using a BCA protein microassay and also by SDS-PAGE assay (Laemmli, Nature 227:600-605 (1970)). The amount of OVA present was determined against a series of OVA standards in suitable buffers (in triplicate).

Results

The amount of OVA entrapped in each of the formulations was as below:

| | | | | OVA Load |
|---|---|---|---|---|
| | | Encapsulation | OVA Load | (%) |
| | Stabilizer | Efficiency^{(a)} | (%) w/w by | (w/w) by |
| Stabilizer | (%) w/v | (%) | BCA | SDS-PAGE |
| Eudragit | 2.5 | 38.3 | 3.5 | 3.1 |
| Eudragit | 4 | 62.7 | 5.7 | 4.7 |
| Eudragit | 6 | 19.0 | 1.7 | 2.0 |
| CMEC | 4 | 48.1 | 4.4 | 4.4 |
| CMEC | 6 | 30.0 | 2.7 | 1.6 |
| CMEC | 8 | 34.4 | 3.1 | 2.4 |

(a) The encapsulation efficiency is defined as the quantity of material (OVA) encapsulated with respect to the amount in the original aqueous solution used to prepare the initial water in oil emulsion.

### EXAMPLE 3

### Preparation of Microspheres Loaded with a Model Antigen (Ovalbumin)

Microspheres similar to those described in the prior art were prepared as described in Example 1 except that the aqueous phase used as the external phase in the water in oil in water emulsion contained 10% w/v polyvinyl alcohol (PVA) (87-89% hydrolyzed, average molecular weight 13 kD to 23 kD as obtained from Aldrich, Gillingham, UK) as stabilizer instead of an enteric polymer. The resulting microspheres did not, therefore, have an enteric coating and are not part of the present invention, but are used as controls in subsequent examples.

The encapsulation efficiency was 54%. The measured particle diameters were d(50%) 0.49 μm, d(10%) 0.25 μm and d(90%) 0.96 μm, respectively. The OVA loading as measured by BCA assay and SDS PAGE, respectively, were 4.92% w/w and 3.43% w/w.

### EXAMPLE 4

### Surface Localization and Release of Bioactive Materials in Microspheres with Enteric Polymers

Methods

The release of OVA from the microparticles was evaluated. OVA-loaded microparticles were incubated for 1 hour in acid medium, (0.5 ml, 0.7% v/v HCl+0.2% w/v NaCl aqueous solution, pH 1.2) at 37° C. to simulate the stomach. The microparticles were then isolated by centrifugation and re-suspended in pH 7.4 phosphate buffered saline (PBS) at 37° C. to simulate the intestines. At intervals over a 7 day period, samples of PBS were removed and assayed for OVA content. Fresh medium was added to each sample of microparticle suspension to replace the volume removed. The acid and PBS samples were analyzed for OVA content by a BCA assay.

The level of surface-located OVA was measured by incubating the microparticles in 0.5 ml PBS, pH 7.4 containing 30 μg pepsin or in simulated gastric fluid (pH 1.2, 3.2 mg/ml pepsin). The level of OVA was assayed via BCA and its structural integrity via SDS-PAGE, plus Western Blotting where appropriate.

Results

The release studies showed that OVA was not released from microparticles stabilized with enteric polymer (CMEC) when incubated for 1 hr at pH 1.2. In contrast, microparticles prepared using a non-enteric stabilizer (polyvinyl alcohol, PVA) released 13.5% of the total OVA content at pH 1.2. When the medium was changed to pH 7.4 PBS, only an additional 5% OVA was released after 2 days from the PVA-stabilized microparticles. In contrast, more than 15% of the OVA load was released from the CMEC-stabilized microparticles within the same time period at pH 7.4.

Treatment of CMEC-stabilized microparticles with pepsin resulted in the loss of some OVA (measured by BCA), but significantly less than that observed with PVA-stabilized microparticles. This demonstrated that the surface layer of enteric polymer could substantially protect an attached antigen from disadvantageous modification by the pH and enzymes present in the stomach of an animal.

| | | Loss of microsphere- |
|---|---|---|
| | | associated OVA(%) after |
| Stabilizer | Stabilizer (%) w/w | incubation with pepsin |
| CMEC | 4 | 4.0 |
| CMEC | 6 | 4.4 |
| CMEC | 8 | 13.1 |
| PVA (Not enteric) | 10 | 44.7 |

The microparticle morphology was maintained after incubation of the CMEC stabilized microspheres in pepsin and gastric media as assessed by electron microscopy.

The percentage of OVA remaining intact after treatment with simulated gastric fluid was determined by SDS-PAGE. It was found that following treatment with the simulated gastric fluid, a greater percentage of intact OVA was present in the CMEC-stabilized microspheres (33-61%) than in microspheres stabilized with the non-enteric polymer PVA (less than 30% intact OVA). Western Blotting showed that when using the enteric polymers as a microsphere stabilizer, an increased amount of intact and antigenic OVA was associated with the microspheres (over formulations prepared without enteric polymer).

These data indicate that the stabilization of microspheres with enteric polymers confers a significant degree of protection of antigenic agents as compared to equivalent systems prepared with a non-enteric stabilizing agent.

### EXAMPLE 5

### Surface Localization of Enteric Polymers on the Microspheres

Methods

The surface localization of the stabilizing enteric polymers was measured using two different techniques. Standards of enteric polymers, OVA and other stabilizers were appropriately made.

The surface localization of the two enteric polymers, CMEC and Eudragit L-100-55, and polyvinyl alcohol (PVA) (control) microspheres was determined by X-ray photoelectron spectroscopy (XPS) and static secondary ion mass spectroscopy (SSIMS). XPS spectra were acquired using a VG Scientific ESCALAB Mark II instrument employing Mg Kα X-rays and electron take-off angles of 35° and 65° relative to the sample surface giving analysis depths of 3 mm and 5 mm. The X-ray gun was operated at 10 KeV and 20 mA. Survey spectra were run with a pass energy of 50 eV and high resolution. Peak areas were calculated after subtraction of linear background and spectra fitted with Gaussian peaks with 20% Lorentzian character.

SSIMS spectra were collected using a VG lonex SIMSLAB3B instrument equipped with a differentially pumped EX05 ion gun and a 12-12M quadrapole mass spectrometer. An argon atom beam was utilized with a total dose per sample below a 10¹³ atoms/cm² threshold.

Results

XPS showed that the surfaces of the microspheres were well covered with enteric polymers but the coverage was not 100% complete. SSIMS showed that use of the two enteric polymers as stabilizers reduced the amount of surface located OVA and effectively covered what was present as compared to microspheres made with PVA.

### EXAMPLE 6

### Improvements in Performance of Microspheres made with Enteric Polymer Stabilizers

Methods

The in vivo performance of the antigenic agent OVA (0.1 mg) adsorbed and entrapped within the microspheres stabilized with enteric polymers, was assessed in an immunogenicity model in mice. Groups of 8 week old female BALB/c mice (n=8) were immunized by oral gavage on three consecutive days with 0.1 mg OVA as follows:

1. Microspheres with 10% w/v PVA as stabilizer. (Control)

2. Microspheres with 4% w/v Eudragit L-100-55 as stabilizer.

3. Microspheres with 4% w/v CMEC as stabilizer.

The microspheres were prepared as in Example 1 (for microsphere systems 2 and 3) and as in Example 3 (for microsphere system 1). Doses were administered in a volume of 0.5 ml distilled water. An identical series of booster immunizations was carried out 4 weeks later. Blood and saliva were collected by approved methods prior to immunization, at 4 weeks following primary immunization and at 2 and 4 weeks following booster immunization. Serum was collected by centrifugation and stored until required. Saliva was collected according to the same schedule as above. Specific IgG and IgA anti-OVA antibodies generated in the mice were detected by a specific ELISA assay, as known to the person skilled in the art. Mean values were compared using an unpaired Students t-test to assess statistical significance. Results were considered significant if p<0.05.

Results

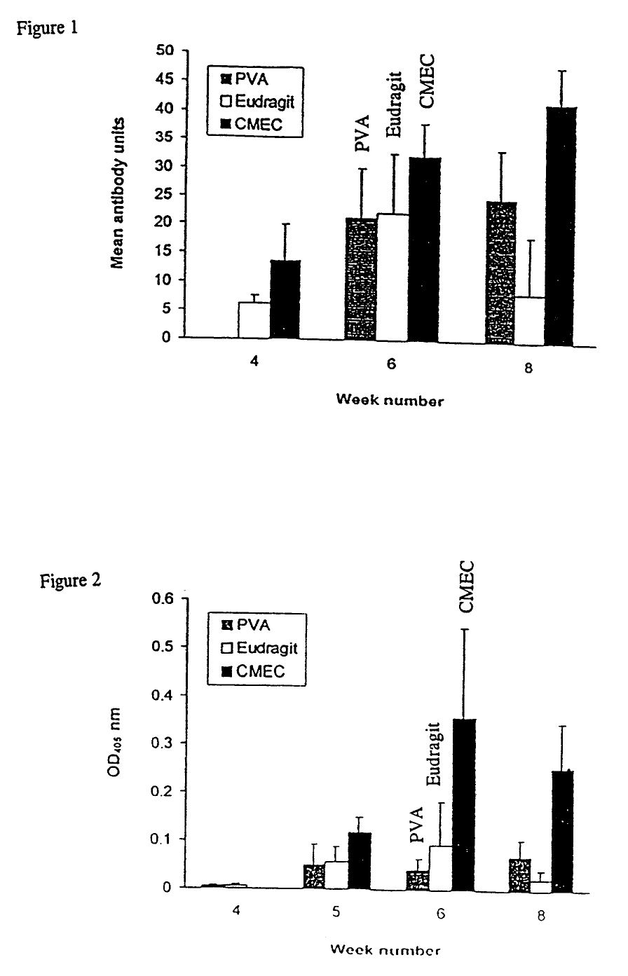

The levels of specific IgG anti-OVA antibodies detected in the serum were raised after booster immunization (see FIG. 1). IgG levels are expressed in mean antibody units. The levels of specific IgG elicited to OVA associated with the CMEC formulation were significantly higher than the other formulations at week 8. The levels of specific IgA anti-OVA antibodies detected in saliva were raised after booster immunization (FIG. 2). IgA levels obtained by ELISA are expressed as optical density measurements at a wavelength of 405 nm (Titertek multiscan ELISA reader). The highest levels were detected in mice immunized with microspheres stabilized with CMEC. CMEC microspheres induced antibody levels in saliva that were significantly higher (p<0.05) than those elicited after immunization with microspheres not stabilized with enteric polymers (PVA). Two weeks after boosting, anti-OVA levels were 9-fold higher with CMEC microspheres than the levels found for PVA microspheres.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I claim:

1. A microparticle composition comprising a biodegradable synthetic polymer microparticle, a proteinaceous antigen and an enteric polymer, wherein the enteric polymer forms a coating layer on a surface of the microparticle.

2. The microparticle composition according to claim 1, wherein the enteric polymer is a stabilizer of the microparticles during preparation thereof.

3. The composition according to claim 1, wherein the enteric polymer comprises at least one of carboxymethylethylcellulose, hydroxpropylmethylcellulosephthalate and cellulose acetate phthalate.

4. The composition according to claim 1, wherein the enteric polymer comprises a methacrylic acid polymer.

5. The composition according to claim 1, wherein the biodegradable synthetic polymer comprises a polylactide-co-glycolide.

6. The composition according to claim 1, wherein the biodegradable synthetic polymer is selected from the group consisting of polylactides, polycaprolactones, polyhydroxyalkanoates, polyorthoesters, polyanhydrides, polyphosphazenes, polyalkylcyanoacrylates, polymalic acids, polyacrylamides, polylactide-polyethylene glycol copolymers, and polycarbonates.

7. The composition according to claim 1, which is adapted for mucosal delivery.

8. The composition according to claim 7, which is adapted for oral delivery.

9. The composition according to claim 1, wherein the biodegradable microparticle is made from a material selected from the group consisting of polylactic acid, polyglycolic acid, and copolymers of these two materials (polylactide-co-glycolides).

10. A composition according to claim 9, wherein the molar ratio of lactide:glycolide units in the copolymer ranges from 10:90 to 90:10.

11. A composition according to claim 1, wherein the microparticles have a diameter less than 1000 μm.

12. A process for preparing a microparticulate composition comprising polymeric microparticles formed from a biodegradable synthetic polymer, a proteinaceous antigen carried by the microparticles and a coating of an enteric polymer on a surface of the microparticles, which process comprises forming the polymeric microparticles in the presence of the antigen and the enteric polymer.

13. The process as claimed in claim 12, wherein the process comprises an emulsification process.

14. The process as claimed in claim 13, wherein the process comprises a water in oil in water emulsification process in which the enteric polymer acts as a stabilizer for the microparticles which are formed in the process.

15. The process as claimed in claim 13, which comprises a double or single-emulsification process in which the biodegradable polymer is dissolved in a suitable solvent and then emulsified using an aqueous solution of the enteric polymer.

16. The process as claimed in claim 12, which results in a microparticulate formulation in which the microparticles have a size range of 200 nm to 1000 μm.

17. A method of enhancing the delivery of an oral or mucosal vaccine which comprises using a microparticle composition according to claim 1 to deliver the vaccine to an animal.

18. A microparticle composition comprising a biodegradable synthetic polymer microparticle, a proteinaceous antigen and an enteric polymer, wherein the enteric polymer forms a coating layer on a surface of the microparticle, and wherein the microparticle is formed in the presence of the antigen and the enteric polymer.

19. The microparticle composition as claimed in claim 18, wherein the microparticle is formed by an emulsification process.

20. The microparticle composition as claimed in claim 19, wherein the microparticle is formed by a water-in-oil-in-water emulsification process in which the enteric polymer acts as a stabilizer for the microparticle formed in the process.

21. The microparticle composition as claimed in claim 19, wherein the microparticle is formed by a double or single-emulsification process in which the biodegradable polymer is dissolved in a suitable solvent and then emulsified using an aqueous solution of the enteric polymer.
